(19) United States
(12) Patent Application Publication
(10) Pub. No.: US 2008/0213851 A1
(43) Pub. Date: Sep. 4, 2008

(54) Enzymatic Method for Producing Bioactive, OsteoblastStimulating Surfaces and Use Thereof

(76) Inventors: Werner E.G. Muller, Wiesbaden (DE); Heinz C. Schroder, Wiesbaden (DE)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 11/579,020

(22) PCT Filed: May 2, 2005
(86) PCT No.: PCT/EP05/04738

§ 371 Date: Sep. 13, 2007

(30) Foreign Application Priority Data
Apr. 30, 2004 (DE) 10 2004 021 229.5

(51) Int. Cl. C12P 3/00; C01B 33/20; C12N 5/06

(52) U.S. Cl. 435/168; 423/325; 435/402

(57) ABSTRACT

The invention relates to a method for producing bioactive surfaces by enzymatic modification of molecules or molecular aggregates, in particular, collagen, on surfaces of glass, metals, metallic oxides, plastics, biopolymers or other materials with an amorphous silicon dioxide (silica) or silicones in the cell culture, by tissue engineering or in medical implants, whereby a polypeptide is used for enzymatic modification, which contains a silicatein α or silicatein β domain. The inventive method promotes the growth, activity and/or mineralization of cells/cell cultures.

### 1. STATE OF THE ART

Silicon dioxide, silicates and silicones are widely used and economically significant materials in industry. They also belong to the main materials used to produce high-technology products (such as optical and microelectronic instruments, production of nanoparticles). Silicon dioxide (SiO₂) occurs in crystalline and in amorphous form. Amorphous SiO₂ is used, among other things, as a molecular sieve, as catalyst, filler, whitening agent, for adsorption, as carrier, stabilizer or carrier for catalysts. Amorphous SiO₂ (“biosilica”) is also the material of which the skeletal structures, formed by biomineralization, of many mono-cellular and multi-cellular organisms consist, such as the shells of siliceous algae (diatoms) and the needles (spicules) of siliceous sponges.

The chemical synthesis of polymeric silicates usually requires drastic conditions such as high pressure and high temperature. In contrast thereto, siliceous sponges are capable, with the aid of specific enzymes, of forming silicate skeletons under mild conditions, that is, at relatively low temperature and low pressure. The SiO₂ synthesis in these organisms is distinguished by high specificity, ability to be regulated and the ability to synthesize defined nanostructures.

First insights into the mechanisms that participate in the formation of biogenic silica could be obtained in the last few years. It surprisingly turned out that siliceous sponges are capable of enzymatically synthesizing their silica skeleton. This became clear by the isolation of the first genes and proteins that participate in the formation of silicon dioxide.

The formation of spicules in demosponges begins around an axial filament that consists of a protein (“silicatein”), is enzymatically active and mediates the synthesis of amorphous silicon dioxide (Cha at al. (1999) Proc. Natl. Acad. Sci. USA 96:361-365; Krasko et al. (2000) Europ. J. Biochem. 267:4878-4887). The enzyme was cloned from the marine siliceous sponge Suberites domuncula (Krasko et al. (2000) Europ. J. Biochem 267:4878-4887) and its technical used described; the first enzyme described is a silicatein α (also named simply silicatein) (PTC/US 30601. Methods, compositions, and biomimetic catalysts, such as silicateins and block copolypeptides, used to catalyze and spatially direct the polycondensation of silicon alkoxides, metal alkoxides, and their organic conjugates to make silica, polysiloxanes, polymetallo-oxanes, and mixed poly(silicon/metallo)oxane materials under environmentally benign conditions. Inventors/applicants: D E Morse, G D Stucky, T D Deming, J Cha, K Shimizu, Y Zhou; DE 10037270 A1. Silicatein-vermittelte Synthese von amorphen Silicaten und Siloxanen und ihre Verwendung. German Patent Office 2000. Applicants and inventors: WEG Müller, B Lorenz, A Krasko, H C Schröder; PCT/EP01/08423. Silicatein-mediated synthesis of amorphous silicates and siloxanes and use thereof. Inventors/applicants: W E G Müller, B Lorenz, A Krasko, H C Schröder). It is capable of synthesizing biosilica from organic silicon compounds (alkoxysilanes).

Silicatein β has also been cloned in addition to silicatein α (DE 103 52 433.9. Enzymatische Snthese, Modifikation und Abbau von Silicium(IV)-und anderer Metall(IV)-Verbindungen. German Patent Office 2003. Applicant: Johannes Gutenberg University Mainz; inventors: W E G Müller, H Schwertner, H C Schröder).

The silicateins are representatives of the cathepsin family. Just as in the cathepsins, e.g., from higher vertebrates the amino acids Cys, His and Asn, that form the catalytic triad (CT) of cysteine proteases, are present in the sponge cathepsins (derived amino acid sequences of the cathepsin L-cDNAs of the sponges Geodia cyclonium and S. domuncula); however, in silicatein α and silicatein β (S. domuncula) the cysteine group is replaced by serine (Krasko et al. (2000) Europ. J. Biochem. 267:4878-4887).

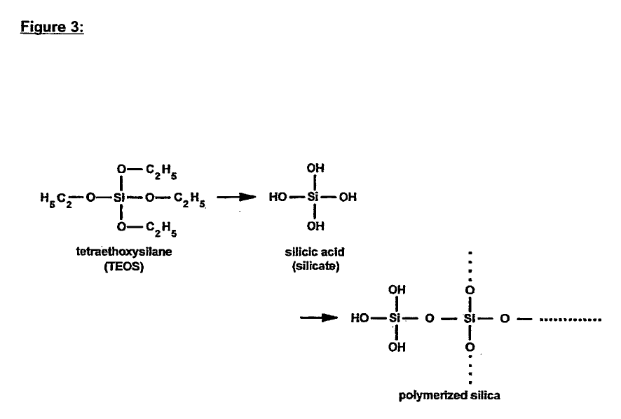

In order to measure the enzymatic activity of recombinant silicateins tetraethoxysilane is customarily used as substrate, wherein the silanols produced after the enzyme-mediated splitting off of ethanol polymerizes (FIG. 3). The amount of polymerized silicon dioxide can be determined with the aid of a molybdate assay (Cha et al. (1999) Proc. Natl. Acad. Sci. USA 96:361-365; Krasko et al. (2000) Europ. J. Biochem. 267:4878-4887).

It was also possible to clone an enzyme from S. domuncula that is capable of dissolving amorphous silicon dioxide (H C Schröder, A Krasko, G Le Pennec, T Adell, M Wiens, H Hassanein, I M Müller, W E G Müller (2003) Silicase, an enzyme which degrades biogenous amorphous silica: Contribution to the metabolism of silica deposition in the demosponge Suberites domuncula. Prog. Molec. Subcell. Biol. 33:250-268; DE 102 46 186.4. Abbau und Modifizierung von Silicaten und Siliconen durch Silicase und Verwendung des reversiblen Enzyms. German Patent Office 2002. Applicant: Johannes Gutenberg University Mainz; inventors: W E G Müller, A Krasko, H C Schröder). The silica-degrading enzyme, silicase, was identified using the technology of differential display of the mRNA. Silicase codes for the one carbonic anhydrase-like enzyme. Recombinant silicase brings about the dissolution of silicon dioxide under the formation of free silicic acid. However, the enzyme is also capable of its synthesis in the reversible reaction. Northern blot experiments showed that in S. domuncula that when the concentration of silicon is elevated in the medium the expression of the silica-anabolic enzyme, silicatein, as well as that of the silica-catabolic enzyme, silicase, rises.

### 1.1. Osteoblasts

Osteoblasts are bone-forming cells. They synthesize and secrete most of the proteins of the bone matrix, including type I collagen and non-collagen proteins. They have a high content of alkaline phosphatase that participates in the mineralization. Osteoblasts react to 1α25-dihydroxyvitamin D_{3 [}1.25(OH)₂D₃], glucocorticoids and growth factors. 1.25(OH)₂D₃ is a stimulator of bone resorption; in mature osteoblasts it increases the expression of genes such as osteocalcin that are associated with the mineralization process.

Typical markers for the osteoblast phenotype are, among others, alkaline phosphatase, osteocalcin, type I collagen, fibronectin, osteonectin, sialoprotein, proteoglycans and collagenase. Alkaline phosphatase is an ectoenzyme (an enzyme oriented from the cell outward) that is bound to the membrane via a glycosylphosphatidylinositol anchor.

There are a number of osteoblast cell lines. SaOS-2 cells are an established human osteosarcoma cell line used as experimental model for studying the function of osteoblasts. They are probably the most-differentiated osteoblast-like cells among the available human cell lines (Rifas et al. (1994) Endocrinology 134:213-221). SaOS-2 cells have a high alkaline phosphatase activity, osteonectin as well as parathomone and 1.25(OH)₂D₃ receptors and are capable of mineralizing (Rodan et al. (1987) Cancer Res. 47:4961-4966; McQuillan et al. (1995) Bone 16: 415-426). The collagen synthesized for the construction of the matrix consists primarily of type I and type V collagen.

The mineralization of osteoblast cultures such as SaOS-2 is furthered by the addition of β-glycerophosphate. β-Glycerophosphate is split by the outwardly oriented alkaline phosphatase, inorganic phosphate (Pᵢ) being released. Ascorbic acid is also frequently added for the mineralization in order to further the formation of the collagen matrix, on which the hydroxylapatite crystals can settle (McQuillan et al. (1995) Bone 16:415-426). The mineralization can be readily demonstrated 6 to 7 days after confluence in stimulated SaOS-2 cells.

The mechanism of osteoblast adhesion to the extracellular matrix of the bone is complex. The adhesion to the collagen substrate seems to regulate the osteoblast differentiation and osteoblast function. For example, peptides containing the Arg-Gly-Asp (RGD) motive block the mineralization and subsequent osteoclast development in rat osteoblasts but have no influence on the collagen synthesis by these cells (Gronowicz and Derome (1994) J. Bone Miner. Res. 9:193-201). On the other hand, it has been shown that surfaces with RGD tripeptides further the osteoblast activity (El-Ghannam et al. (2004) J. Biomed. Mater. Res. 68A:615-627).

Interactions of integrins with extracellular matrix proteins decisively participate in the mechanism of adhesion and in the following cellular processes. Human osteoblasts express a plurality of integrins. It has been shown that certain integrins play a part in the induction of the expression of alkaline phosphatase by interleukin-1 in human MG-63 osteosarcoma cells (Dedhar (1989) Exp. Cell. Res. 183: 207-204). Other integrins have been identified as adhesion receptors for collagen (Hynes (1992) Cell 69:11-25). In this manner, the tetrapeptide motive Asp-Gly-Glu-Ala (DGEA) (Staatz et al. (1991) J. Biol. Chem. 266:7363-7367) contained in the type I collagen is recognized by an integrin expressed by human osteoblasts (Clover et al. (1992) J. Cell Sci. 103:267-271). The DGEA peptide also brings about a rise of Ca²⁺ in SaOS-2 cells (McCann et al. (1997) Matrix Biol. 16:271-280).

### 2. SUBJECT MATTER OF THE INVENTION

There is a great need for alternative bone replacement materials due to the disadvantages of autotransplants that are preferably used with preference in the clinic for bone repair and bone replacement. In orthopedics biodegradable polymers such as polylactides (PLA), polyglycolides (PGA) and their copolymers (PLAGA) are frequently used. In recent years, so-called bioactive materials such as 45S5 bioactive glass have been developed that stimulate the new formation of bone and build up a continuous connection to the bone via a calcium phosphate layer on their surface (Hench et al. (1991) J. Amer. Cerm. Soc. 74:1487). However, this does not make a non-physiological surface matrix (glass-surface) available.

Therefore, a problem of the present invention is to make suitable physiological surfaces available with properties that are improved in comparison to the traditionally used materials.

This problem is solved in accordance with a first aspect of the invention by the surface matrix in accordance with the invention and consisting of physiological molecules/molecular aggregates (collagen) and modified with enzymatically produced biosilica.

In the framework of the invention a material is designated as “bioactive” when a specific biological response is produced on its surface that ultimately results in the formation of a (stable) bond between the material and the tissue (such as, e.g., new bone formation). Thus, a “bioactive” material contributes to the furthering of cell growth and/or cell differentiation and/or the modulation of specific cell functions (such as the furthering of the mineralization by osteoblasts or the furthering of collagen formation by fibroblasts and/or further cell functions).

It has been shown that the expression of type I collagen, of alkaline phosphatase as well as of bone morphogenetic protein-2 (BMP-2) is elevated in vitro by surface-active glasses (bioactive glasses) (Gao et al. (2001) Biomaterials 22:1475-1483; Bosetti et al. (2003) J. Biomed. Mater. Res. 64A:189-95).

Silicic acid plays an important part in bone formation. Thus, it is known that orthosilicic acid stimulates the type 1 collagen synthesis and the differentiation in human osteoblasts in vitro (Reffitt et al. (2003) Bone 32:127-135). Likewise, the alkaline phosphate activity and osteocalcin are also significantly raised.

The following are indicated as survey articles for clinical applications of bioactive glasses and glass ceramics: Gross et al. (1988) CRC Critical Reviews in Biocompatibility 4:2; Yamamuro et al. (editors), Handbook on Bioactive Ceramics, vol. I: Bioactive Glasses and Glass-Ceramics, vol. II. CRC Press, Boca Raton, Fla., 1990; Hench and Wilson (1984) Science 226:630.

Prerequisites for such bone replacement materials are that they are biocompatible, biodegradable and osteoconductive (capable of promoting bone growth), that is, bioactive (are capable of forming a calcium phosphate layer on their surface, see above).

Therefore, according to a further aspect of the present invention a method for producing bioactive surfaces by enzymatic modification of molecules or molecular aggregates on surfaces with amorphous silicon dioxide (silica) is described wherein a polypeptide is used for the enzymatic modification, characterized in that the polypeptide contains an animal, bacterial, vegetable or fungal silicatein α silicatein β domain exhibiting at least 25%, preferably at least 50%, more preferably at least 75% and most preferably at least 95% sequence identity with the sequence shown in SEQ ID No. 1 or SEQ ID No. 3.

It was previously not known and could not be recognized from the state of the art that it is possible to obtain bioactive surfaces by enzymatic modification of molecules or molecular aggregates on surfaces with amorphous silica dioxide (silica).

Therefore, a method in accordance with the invention is also made available that is characterized in that compounds such as silicic acid, monoalkoxysilanetriols, dialkoxysilanediols, trialkoxysilanols or tetraalkoxysilanes are used as substrate for the enzymatic modification.

According to a further aspect of the present invention the method can also serve to produce bioactive surfaces by enzymatic modification of molecules or molecular aggregates on surfaces with silicones, where a polypeptide is also used for the enzymatic modification that is characterized in that it contains an animal, bacterial, vegetable or fungal silicatein α or silicatein β domain exhibiting at least 25%, preferably at least 50%, more preferably at least 75% and most preferably at least 95% sequence identity with the sequence shown in SEQ ID No. 1 or SEQ ID No. 3.

Compounds such as monoalkoxysilanediols, monoalkoxysilanols, dialkoxysilanols, alkylsilanetriols, arylsilanetriols or metallosilanetriols, alkylsilanediols, arylsilanediols or metallosilanediols, alkylsilanols, arylsilanols or metallosilanols, alkylmonoalkoxysilanediols, arylmonoalkoxysilanediols or metallomonoalkoxysilanediols, alkylmonoalkoxysilanols, arylmonoalkoxysilanols or metallomonoalkoxysilanols, alkyldialkoxysilanols, aryldialkoxysilanols or metallodialkoxysilanols, alkyltrialkoxysilanes, aryltrialkoxysilanes or metallotrialkoxysilanes can be used for the last-named aspect (production of bioactive surfaces by enzymatic modification of molecules or molecular aggregates of surfaces with silicones) as substrate for the enzymatic modification. According to yet another aspect of the present invention, the method can also serve to produce bioactive surfaces by enzymatic modification of molecules or molecular aggregates on surfaces with amorphous silicon dioxide (silica), where a polypeptide is also used for the enzymatic modification that is characterized in that it contains an animal, bacterial, vegetable or fungal silicatein α or silicatein β domain exhibiting at least 25%, preferably at least 50%, more preferably at least 75% and most preferably at least 95% sequence identity with the sequence shown in SEQ ID No. 5.

According to another aspect of the present invention a production of bioactive surfaces by enzymatic modification of molecules or molecular aggregates on surfaces of glass, metals, metal oxides, plastics, biopolymers or other materials can take place by the method in accordance with the invention.

According to yet another aspect of the present invention a method for the production of bioactive surfaces by enzymatic modification of molecules or molecular aggregates on surfaces is made available, wherein the molecules or molecular aggregates are biopolymers, especially collagen, and preferably collagens from a marine sponge.

Furthermore, a method in accordance with the invention for promoting the growth, activity and/or the mineralization of cells/cell cultures, especially osteoblasts, is made available in which a) molecules or molecular aggregates on surfaces with amorphous silicon dioxide (silica) are enzymatically modified and b) a polypeptide is used for the enzymatic modification, that is characterized in that the polypeptide contains an animal, bacterial, vegetable or fungal silicatein α or silicatein β domain exhibiting at least 25%, preferably at least 50%, more preferably at least 75% and most preferably at least 95% sequence identity with the sequence shown in SEQ ID No. 1 or SEQ ID No. 3.

A polypeptide can also be used in the method in accordance with the invention for promoting the growth, activity and/or the mineralization of surfaces with amorphous silicon dioxide (silica) that is characterized in that the polypeptide comprises an animal, bacterial, vegetable or fungal silicase domain exhibiting at least 25%, preferably at least 50%, more preferably at least 75% and most preferably at least 95% sequence identity with the sequence shown in SEQ ID No. 5.

The previously described method in accordance with the invention is used in cell culture, tissue engineering or in the production of medical implants.

A further aspect of the present invention concerns a structure or surface that contains silicic acid and that was obtained in accordance with the method of the invention.

The polypeptide used in accordance with the invention (silicatein α or silicatein β from Suberites domuncula in accordance with SEQ ID No. 1 or SEQ ID No. 3 or a polypeptide homologous to it that exhibits at least 25%, preferably at least 50%, more preferably at least 75% and most preferably at least 95% sequence identity with the sequence shown in SEQ ID No. 1 or SEQ ID No. 3 in the amino acid sequence of silicatein α or silicatein β) can, in addition to the natural form, be further characterized in that it was synthetically produced or in that the polypeptide is present in a prokaryotic or eukaryotic cell extract or cell lysate. The cell extract or the lysate can be obtained from a cell ex vivo or ex vitro, e.g., from a recombinant bacterial cell or a marine sponge. In the case of the polypeptide used in accordance with the invention it can also be a silicase from Suberites domuncula according to SEQ ID No. 5 or a polypeptide homologous to it that exhibits at least 25%, preferably at least 50%, more preferably at least 75% and most preferably at least 95% sequence identity with the sequence shown in SEQ ID No. 5 in the amino acid sequence of the silicase domain.

The polypeptide used in accordance with the invention can be purified according to traditional methods known in the state of the art and thus be present substantially free of other proteins.

The properties of the cDNAs coding for the silicatein α polypeptide and the silicatein β polypeptide from S. domuncula as well as the polypeptides derived from the nucleotide sequence have been described (PCT/US99/0601; DE 10037270 A 1; PCT/EP01/08423; DE 103 52 433.9). The molecular weight of the recombinant silicatein α polypeptide is ˜28.5 kDA (˜26 kDA silicatein plus 2 kDA vector); the isoelectric point is approximately pl 6.16.

The properties of the cDNA coding for the silicase from S. domuncula as well as the polypeptide derived from the nucleotide sequence have also been described (DE 102 46 186.4).

The invention will now be illustrated further by the following examples without being limited by them. The attached figures and the SEQ IDs show:

SEQ ID No. 1: The amino acid sequence of the silicatein α polypeptide from S. domucula used in accordance with the invention.

SEQ ID No. 2: The nucleic acid sequence of the silicatein α polypeptide from S. domuncula used in accordance with the invention.

SEQ ID No. 3: The amino acid sequence of the silicatein β from S. domuncula (SIA_SUBDO) used in accordance with the invention.

SEQ ID No. 4: The nucleic acid sequence of the silicatein β from S. domuncula used in accordance with the invention.

SEQ ID No. 5: The amino acid sequence of the silicase from S. domuncula used in accordance with the invention.

SEQ ID No. 6: The nucleic acid sequence of the cDNA of the silicase from S. domuncula used in accordance with the invention.

SEQ ID No. 7: The amino acid sequence of the collagen 3 from S. domuncula (SIA_SUBDO) used in accordance with the invention.

SEQ ID No. 8: The nucleic acid sequence of the collagen 3 from S. domuncula used in accordance with the invention.

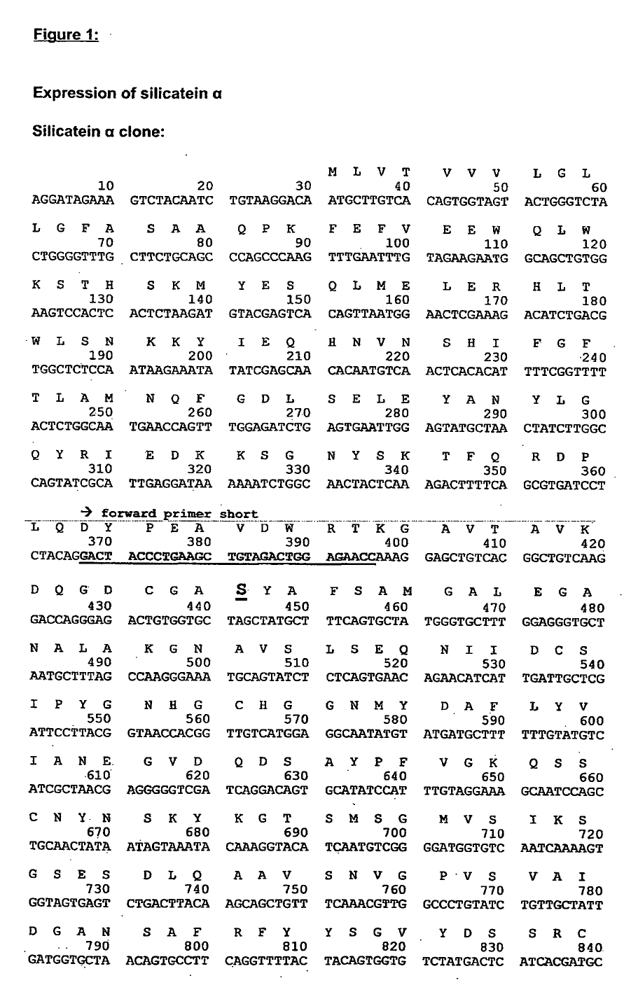

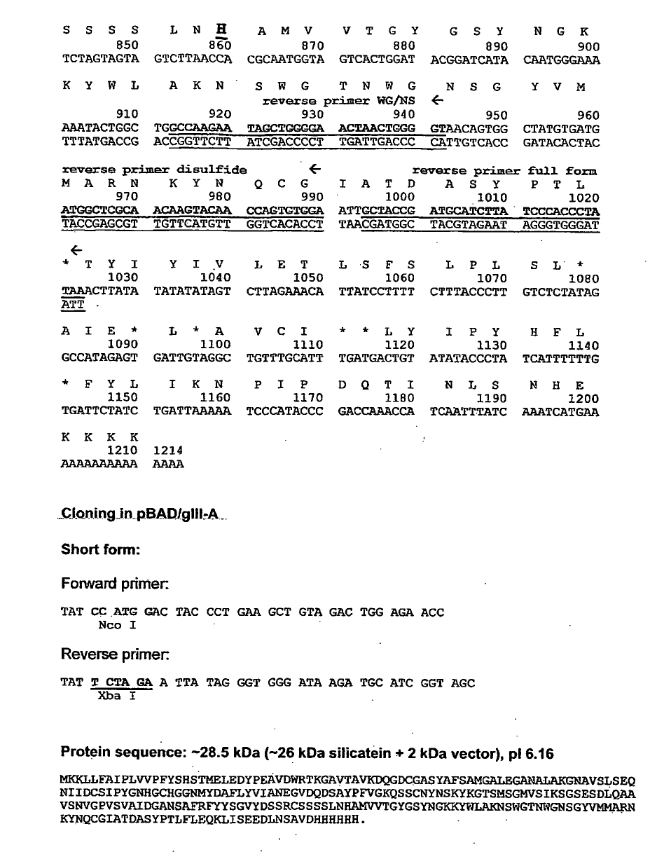

### FIG. 1:

Expression of silicatein α from S. domuncula. The nucleotide sequence of the silicatein α clone (S. domuncula) as well as forward primer and reverse primer for the amplification of the cDNA coding for the short silicatein α form for cloning into the expression vector pBAD/gIII-A and amino acid sequence of the recombinant protein (short form of silicatein α), which amino acid sequence is derived from the nucleotide sequence.

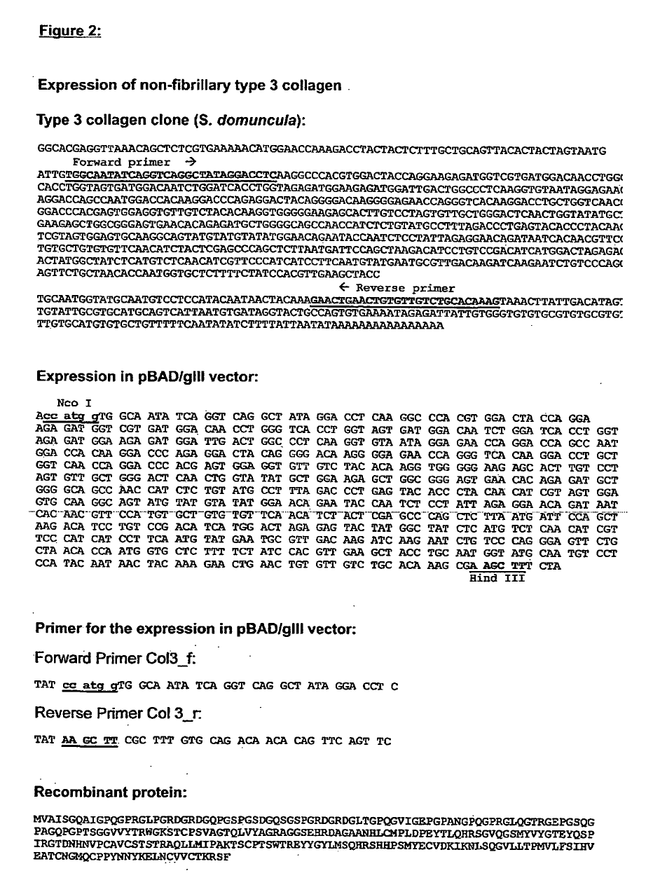

### FIG. 2:

Expression of non-fibrillary type 3 collagen from S. domuncula. The nucleotide sequence of the type 3 collagen clone (S. domuncula) as well as forward primer Col3_f and reverse primer Col3_r for the amplification of the cDNA coding for type 3 collagen for cloning into the expression vector pBAD/gIII-A (the restriction sites of NcoI and HindIII are underlined) and amino acid sequence of the recombinant protein, which amino acid sequence is derived from the nucleotide sequence.

### FIG. 3:

Determination of the silicatein activity. Tetraethoxysilane (TEOS) is usually used as substrate.

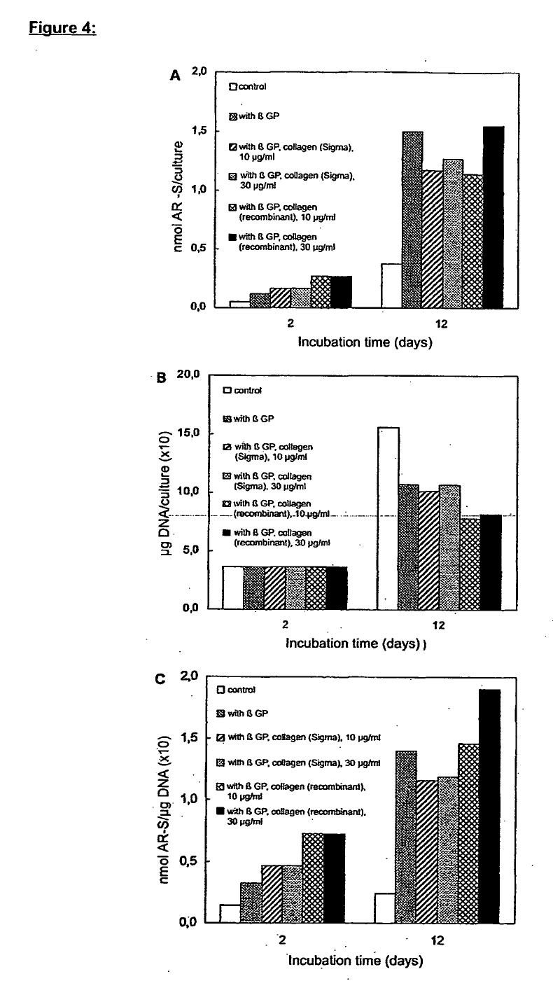

### FIG. 4:

Stimulation of the mineralization of SaOS-2 cells after coating of the culture plates with recombinant non-fibrillary sponge collagen (type 3; S. domuncula) in comparison to fibrillary type 1 bovine collagen (Sigma). The culture plates (24-well plates) were coated with different amounts (10 μg/ml or 30 μg/ml) of either recombinant type 3 collagen (S. domuncula) or type 1 collagen (bovine; Sigma company). Then, the SaOS-2 cells were seeded on the plates and cultivated for 2 and 12 days under standard conditions. β-glycerophosphate (β-GP; 10 mM) was added on day 7 to the batches. Then, the mineralization was determined with alizarin red-S (AR-S; A) as well as the total DNA LB). The mineralization in nmol alizarin red/pμtotal DNA is indicated in (C).

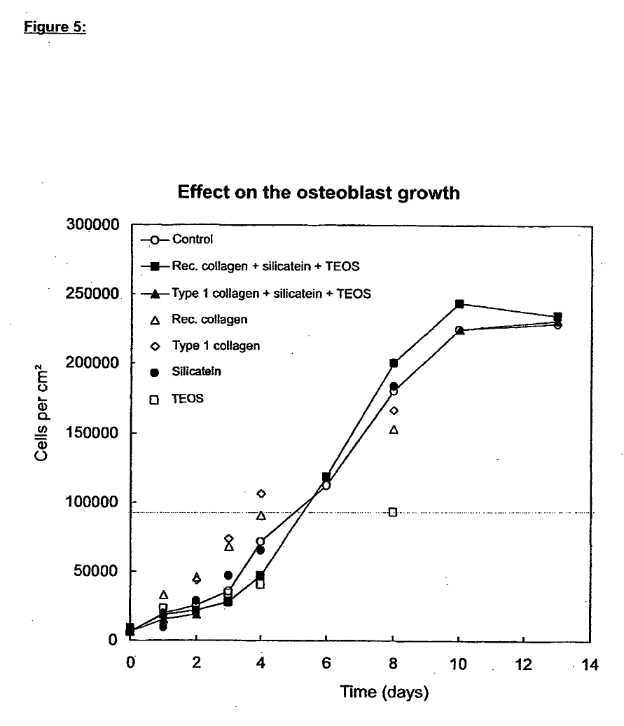

### FIG. 5:

Growth of SaOS-2 cells on the enzymatically modified, osteoblast-stimulating surface in accordance with the invention in comparison to control surfaces (cell density). The results are shown that were obtained with SaOS-2 cells that grew in wells on a non-modified surface (=control) (O), as well as of SaOS-2 cells that grew on surfaces modified in the following manner: (a) modification by coating with recombinant type 3 collagen (S. domuncula) and enzymatically synthesized biosilica (by means of silicatein α and TEOS) (▪), (b) modification by coating with recombinant bovine type 1 collagen and enzymatically synthesized biosilica (by means of silicatein α and TEOS) (▴), (c) modification with recombinant type 3 collagen alone (S. domuncula) (Δ), (d) modification with recombinant bovine type 1 collagen alone (⋄), (e) modification with silicatein alone () and (f) modification by treatment with TEOS without addition of a protein (collagen or silicatein) (□). β-glycerophosphate (10 mM) was added on day 7 to the batches. The cell density (cells per cm²) on day 1, 2, 3, 4, 6 and 8 after the conversion of the cells is indicated.

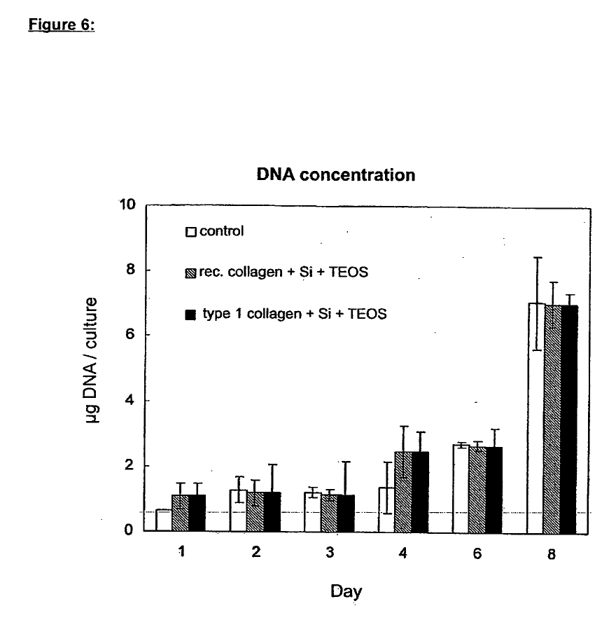

### FIG. 6:

Total DNA amount of SaOS-2 cell cultures on the enzymatically modified, osteoblast-stimulating surface in accordance with the invention in comparison to non-modified control surfaces. The cells grew in wells whose surfaces were modified either with recombinant type 3 collagen (S. domuncula) or type 1 collagen (Sigma), both coated with enzymatically synthesized biosilica (with silicatein α [Si] and [TEOS]) or not. β-glycerophosphate (10 mM) was added to the batches on day 7. The amount of total DNA per culture (well) on day 1, 2, 3, 4, 6 and 8 after the conversion of the cells is indicated.

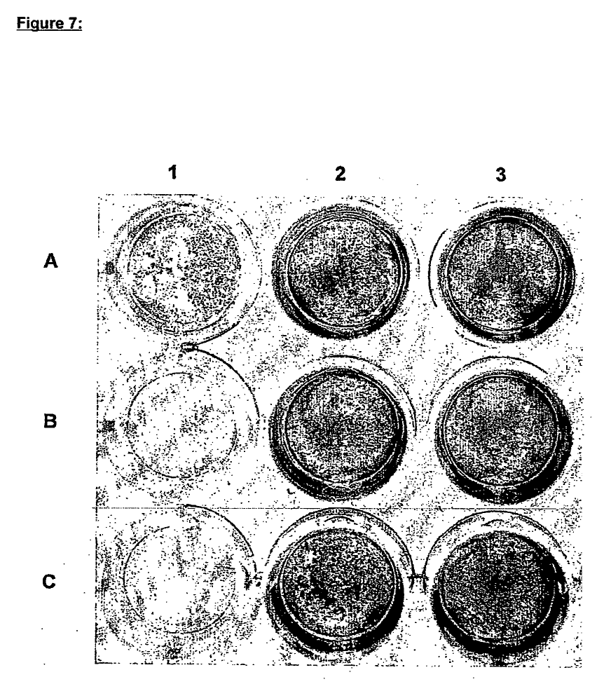

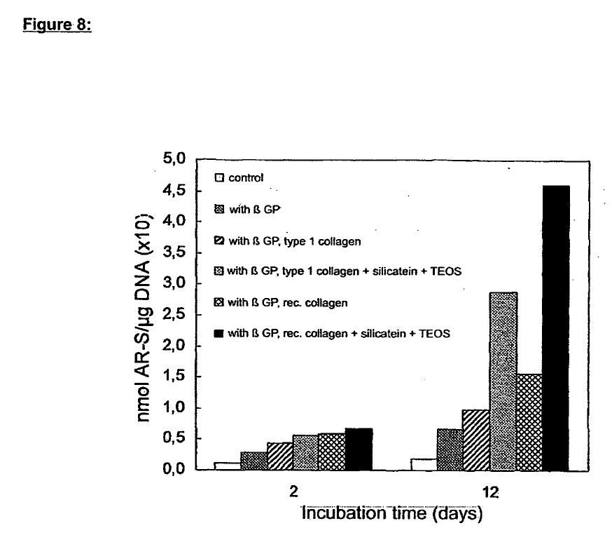

### FIG. 7:

Mineralization of SaOS-2 cells on the enzymatically modified, osteoblast-stimulating surface in accordance with the invention in comparison to non-modified control surfaces. The demonstration of the mineralization took place on day 12 with alizarin red-S. β-glycerophosphate (10 mM) was added to the batches on day 7. 1A: SaOS-2 cells grown on non-modified surface with the addition of β-glycerophosphate from day 7 on (relative strength of the mineralization: ++). 1B, 1C: SaOS-2 cells grown on non-modified surface without the addition of β-glycerophosphate (control; relative strength of the mineralization: +). 2A, 2B, 2C: SaOS-2 cells grown on a modified surface (modification by coating with recombinant sponge type 3 collagen and enzymatically—by means of silicatein α and TEOS—synthesized biosilica), with the addition of β-glycerophosphate (relative strength of the mineralization: +++). 3A, 3B, 3C: SaOS-2 cells with the addition of β-glycerophosphate grown on a modified surface (modification by coating with bovine type 1 collagen and enzymatically—by means of silicatein α and TEOS—synthesized biosilica) (relative strength of the mineralization: +++).

### Illustration 8:

Stimulation of the mineralization of SaOS-2 cells that grew on the enzymatically modified surface in accordance with the invention in comparison to SaOS-2 cells on surfaces after coating with collagen alone and controls (non-coated plates without and with β-glycerophosphate). In order to coat the culture plates either type 1 collagen (bovine; Sigma) alone or recombinant non-fibrillary type 3 collagen (S. domuncula) alone or type 1 collagen plus silicatein α plus TEOS (synthesis of biosilica-modified bovine collagen) or recombinant type 3 collagen plus silicatein α plus TEOS (synthesis of biosilica-modified sponge collagen) was used. Then, the SaOS-2 cells were seeded on the plates and cultivated for 2 and 12 days under standard conditions. β-glycerophosphate (β-GP; 10 mM) was added to the batches on day 7. The mineralization is indicated in nmol alizarin red/μg total DNA.

### EXAMPLES

### 2.1. Mineralization of SaOS-2 Cells on Enzymatically Modified Surfaces

Human osteoblast cells (SaOS-2 cells) were used for the following tests. SaOS-2 cells stem from an osteogenic sarcoma (McQuillan et al. (1995) Bone 16:415-426). The cell growth and the mineralization were determined for all cultures. In addition to the mineralization the expression of the alkaline phosphatase was also measured as a further differentiation marker.

The SaOS-2 cells were cultivated for up to 12 days with 10 mM β-glycerophosphate that had been added on day 7 after the conversion of the cells (start of the experimental cultures). Then, the amount of calcium phosphate deposits was determined in the batches with alizarin red S. The results were related to the total DNA.

The mineralization of the SaOS-2 cells is strongly stimulated by coating the culture plates with collagen (FIG. 4). The recombinant type 3 sponge collagen (S. domuncula) was more efficient in this instance than type 1 bovine collagen (Sigma) (both with an incubation time of 2 days as well as of 12 days if the measured values had been related to μg DNA per batch). The stimulation of the mineralization in the batches with β-glycerophosphate was only approximately equal to that in the wells coated with the type 1 bovine collagen after a longer incubation period (12 days). However, even at this point in time the mineralization was greater than in all other batches for the wells coated with the recombinant sponge collagen.

However, the coating of the plates (wells) with collagen (type 1 bovine collagen as well as recombinant type 3 sponge collagen) had a negative influence on the growth of the SaOS-2 cells (indicated as μg DNA per batch) in a longer incubation period (12 days; FIG. 4).

Analogous results were obtained when the cell density (cells per cm²) was determined (FIG. 5). A distinct stimulation of the growth of the cells was found in the wells (batches) that had been coated with collagen (type 1 bovine collagen or recombinant type 3 sponge collagen) in shorter incubation periods (1 to 4 days), but in a longer incubation period (8 days) the growth was below the control values (wells without collagen coating).

In contrast thereto, higher cell densities, that is, a better growth, than in the controls was found in the wells whose surfaces had been treated with the method in accordance with the invention (modification of the surface with collagen plus enzymatically—by means of silicatein and TEOS—produced collagen) (FIG. 5).

The determination of the concentration of DNA also showed that no reduction of the cell growth (based on the value for the total DNA per culture) occurred in the wells whose surfaces had been treated with the method in accordance with the invention. On day 4 the total DNA in the treated (modified) wells was even higher than in the control (FIG. 6).

The drastic differences between the enzymatically modified, osteoblast-stimulating surface in accordance with the invention in comparison to non-modified control surfaces was apparent in the determination of the mineralization (depositing of calcium phosphate) of the SaOS-2 cells. FIG. 7 shows a demonstration of the mineralization on day 12 with alizarin red-S. On the control surfaces of the non-modified wells, after the addition of β-glycerophosphate (on day 7), there was only a comparatively slight rise of the mineralization (well No. 1A) compared with the controls without β-glycerophosphate (well No. 1B and 1C). In contrast thereto, in the case of SaOS-2 cells that grew on the surface modified by coating with recombinant sponge collagen type 3 and enzymatically—by means of silicatein α and TEOS—synthesized biosilica (well No. 2A, 2B and 2C) as well as in the case of SaOS-2 cells that grew on the surface modified by coating with bovine type 1 collagen and enzymatically—by means of silicatein α and TEOS—synthesized biosilica (well No. 3A, 3B and 3C), a sharp rise in the mineralization was found.

The rise of the mineralization of SaOS-2 cells that grew on the enzymatically modified surface in accordance with the invention (treatment with collagen plus silicatein α plus TEOS) was drastically elevated in comparison to SaOS-2 cells that grew on surfaces that had been modified with collagen alone (illustration 8). As the illustration shows, on day 12 the extent of the mineralization (indicated in nmol alizarin red/μg total DNA) on the culture plates after coating with type 1 collagen plus silicatein α plus TEOS (synthesis of biosilica-modified bovine collagen) or after coating with recombinant type 3 collagen plus silicatein α plus TEOS (synthesis of biosilica-modified sponge collagen) was distinctly above that of the plates coated with the particular collagens alone.

The bioactivity of the enzymatically modified in accordance with the invention can also be demonstrated by measuring the activity of the alkaline phosphatase in mineralized SaOS-2 cells.

### 2.2. Production of Silicatein Polypeptides

The silicatein polypeptides required for the modification of the collagen can be produced from tissues or cells in a purified or recombinant manner.

2.2.1. Purification of the Silicatein Polypeptides from Natural Sources

The purification of silicatein α and silicatein β can be carried out from isolated spicules of sponges.

### 2.2.2. Production of Recombinant Silicatein Polypeptides

The production of the recombinant proteins (silicatein α: SEQ ID No. 1; silicatein β: SEQ ID No. 3) can take place in E. coli. Even a production in yeasts and mammalian cells is possible. To this end the particular cDNA is cloned into an expression vector, e.g., pQE-30. After the transformation of E. coli the expression of the proteins is induced with IPTG (isopropyl-β-thiogalactopyranoside) (Ausubel et al. (1995) Current Protocols in Molecular Biology. John Wiley and Sons, New York). The purification of the recombinant proteins via the histidine tag is carried out on a Ni-NTA matrix.

A sequence corresponding to the enterokinase cleavage site can be introduced between oligohistidine and silicatein. The fusion protein is then cleaved with enterokinase.

Alternatively, e.g., the “GST (glutathione S transferase) fusions” system (Amersham Company) can be used for the expression of the recombinant proteins. Two inserts can be used in order to eliminate potential effects of signal peptides during the expression; one insert comprises the entire derived protein (long form) and the other insert only the active range (short form). The corresponding clones are cloned into plasmid pGEX4T-2 that contains the GST gene of Schistosoma japonicum. After the transformation of E. coli, the expression of the proteins is induced by IPTG. The GTS fusion proteins obtained are purified by affinity chromatography on glutathione sepharose 4B. In order to separate the glutathione-S transferase the fusion proteins are cleaved with thrombin.

Another preferred alternative (used for the experiments described here) is the preproduction of recombinant silicatein α in E. coli using the oligo-histidine expression vector pBAD/gIIIA (Invitrogen) in which the recombinant protein is secreted into the periplasmatic space on the basis of the gene III signal sequence (FIG. 1). The cDNA sequence (SEQ ID No. 2) coding for silicatein α is amplified with PCR using the following primers (short form of silicatein α): Forward primer: TAT CC ATG GAC TAC CCT GAA GCT GTA GAC TGG AGA ACC (SEQ ID No. 9) and reverse primer: TAT T CTA GA A TTA TAG GGT GGG ATA AGA TGC ATC GGT AGC (SEQ ID No. 10); and cloned into pBAD/gIIIA (restriction nucleases for insertion into the expression vector: NcoI and XbaI). After the transformation of E. Coli XL1-blue the expression of the fusion protein is induced with L-arabinose.

The recombinant sponge silicatein polypeptide (short form) has a molecular weight of ˜28.5 kDA (˜26 kDA silicatein plus 2 kDA vector); the isoelectric point is approximately pl 6.16.

Likewise, an insert can also be used that contains the entire derived silicatein a protein (long form).

In an analogous manner, a short and a long form of silicatein β (cDNA: SEQ ID No. 4; amino acid sequence derived from it: SEQ ID No. 3) can be expressed.

### 2.3. Determination of the Silicatein Activity

In order to determine the enzymatic activity of the (recombinant) silicateins an assay can be used that is based on the measurement of polymerized and precipitated silica after hydrolysis and subsequent polymerization of tetraethoxysilane (TEOS) (FIG. 3). Here, the enzyme is usually dissolved in 1 mm of a MOPS buffer (pH 6.8) and compounded with 1 milliliter of a 1-4.5 mM tetraethoxysilane solution. The enzymatic reaction is carried out for times of different lengths usually at room temperature. In order to demonstrate the silica products, the material is centrifuged, washed with ethanol and air-dried. The sediment is subsequently hydrolyzed with 1 M NaOH. The released silicate is quantitatively measured in the produced solution using a molybdate-supported demonstration method (silicon assay of the Merck company).

The hydrolysis of alkoxysilanes by the (recombinant) silicateins can also be determined with the aid of a coupled optical test. This test is based on the determination of the released alcohol. To this end, a solution of ABTS [azino-bis (3-ethylbenzthiazoline-6-sulfonic acid)] in potassium phosphate buffer pH 7.5 (O₂-saturated) as well as a peroxidase solution and an alcohol oxidase solution are pipetted into a cuvefte. H₂O₂ is added after the mixing. After renewed mixing the substrate solution (e.g., tetraethoxysilane [TEOS] in MOPS buffer) or the enzyme (silicatein) in substrate solution is added and the extinction followed in a photometer at 405 nm. Various alcohol (e.g., ethanol) concentrations serve to establish the straight calibration line.

### 2.4. Production of Silicase from Natural Sources and of the Recombinant Enzyme

The purification of silicase from natural sources (such as tissue or cells) and the recombinant production of the enzyme (SEQ ID No. 5) are state of the art (DE 102 46 186.4; PCT/EP03/10983).

### 2.5 Determination of Silicase Activity

The method for demonstrating the silicase activity of (commercial) carbonic anhydrase preparations (e.g., from bovine erythrocytes; Calbiochem company) or of recombinant sponge silicase has been described (DE 102 46 186.4; PCT/EP03/10983).

### 2.6. Production of Sponge Collagen

Both native collagen (from vertebrates such as, e.g., bovine collagen as well as from invertebrates such as, e.g., from marine demosponges) as well as also recombinant collagen (especially from the marine sponge S. domuncula) can be used as template. A few methods for their preparation are described in the following.

### 2.6.1. Isolation of Native Sponge Collagen

A simple method for the isolation of collagen from various marine sponges has been described (DE 100 10 113 A 1. Verfahren zur Isolierung von Schwammkollagen sowie Herstellung von nanopartikulärem Kollagen. Applicant: W Schatton. Inventors: J Kreuter, W E G Müller, W Schatton, D Swatschek, M Schatton; Swatschek et al. (2002) Eur. J. Pharm. Biopharm. 53:107-113). The sponge collagen is obtained with a high yield (>30%).

### 2.6.2. Production of Recombinant Sponge Collagen

In order to produce the recombinant collagen (SEQ ID No. 7), a clone can be used that codes for a non-fibrillary collagen (collagen 3) from S. domuncula.

The cDNA sequence coding for the S. domuncula type 3 collagen (SEQ ID No. 8) can be amplified with PCR using suitable primers and subcloned into a suitable expression vector. The expression was successfully carried out among other things with the bacterial oligo-histidine expression vectors pBAD/gIIIA (Invitrogen) and pQTK_{—}1 (Qiagen) (FIG. 2). The following can be used as primers for the PCR (with subsequent use of pBAD/gIIIA); forward primer: TAT cc atg gTG GCA ATA TCA GGT CAG GCT ATA GGA CCT C (SEQ ID No. 11) and reverse primer: TAT AA GC TT CGC TTT GTG CAG ACA ACA CAG TTC AGT TC (SEQ ID No. 12); restriction nucleases for insertion into the expression vector: NcoI and HindIII. After the transformation of Escherichia coli strain XL1-blue with the plasmid (expression vector) the expression of the fusion protein is induced with L-arabinose (at pBAD/gIIIA) or with isopropyl-β-D-thiogalactopyranoside (IPTG); at pQTK₁₃ 1). The expression vector pBAD/gIIIA has the advantage that the recombinant protein is secreted into the periplasmatic space on the basis of the gene III signal sequence. The signal sequence is removed after the membrane passage. When using pQTK-1 the bacteria are extracted with PBS/8 M urea. The suspension is centrifuged after ultrasonic treatment. The purification of the fusion protein from the supernatant takes place by metal-chelate affinity chromatography using an Ni-NTA agarose matrix (Qiagen) as described by Hochuli et al. (J. Chromatogr. 411: 177-184; 1987). The extract is put on the column; a wash is subsequently performed with PBS/urea and the fusion protein eluted from the column with 150 mM imidazol in PBS/urea.

The molecular weight of the recombinant type 3 collagen (S. domuncula) obtained after expression of the cDNA amplified using the above-mentioned primers is ˜28.5 kDa. The isoelectric point (IEP) of the peptide (see SEQ ID No. 7) derived from the cDNA shown in SEQ ID No. 8 is 8.185. The charge at pH 7.0 is 4.946.

### 2.7. Cell Culture

Human osteosarcoma cells (SaOS-2; American Type Culture Collection) are cultivated in McCoy's medium (Invitrogen) containing 15% fetal bovine serum (FBS) with 1% glutamine, 100 U/ml penicillin, 100 μg/ml streptomycin at 37° C., 98-100% relative humidity and 5% CO₂ atmosphere. The medium is changed every 2 days. In order to produce the experimental cultures, the confluent cells are briefly washed with Hank's balanced saline solution (HBSS) without Ca²⁺ and Mg²⁺ (Sigma) and then trypsinated; treatment with 0.1 wt. % trypsin/0/04 wt. % EDTA in Ca²⁺-free and Mg²⁺-free PBS (137 mM NaCl, 2.7 mM KCl, 10 mM Na₂HPO₄, 1.76 mM KH₂PO₄, pH 7.40). After the formation of a cell suspension the cells were counted in a hemocytometer and seeded with a density of 1000 cells/mm² in 24 well plates (190 mm²). The cultures are subsequently incubated for up to 14 days in growth medium. The medium was changed every 2 days and every day after a week. On day 7, 10 mM β-glycerophosphate (Sigma) 1 M stock solution was added. The mineralization is stimulated by β-glycerophosphate.

### 2.8. Treatment of the Culture Plates and Performance of the Assay

The culture plates are coated with PBS alone (control) or solutions of the following proteins in PBS:
a) Type 1 collagen (Sigma; 10 μg/cm²) plus silicatein (1 μg/cm²) plus TEOS (5 mM) and
b) Type 3 collagen (10 μg/cm²) plus silicatein (1 μg/cm²) plus TEOS (5 mM).

To this end, the microtiter plates are incubated for 1 hour at 37° C. after the addition of collagen, silicatein and TEOS. The plates are subsequently washed once with PBS and the cells placed in.

Other concentrations of the proteins and of the substrate as well as other incubation times also proved to be suitable.

The concentration of the recombinant type 3 collagen (S. domuncula) in the stock solution (PBS, filtered) is 400 μg/ml. This solution was diluted 1:10 in PBS for coating (10 μg/cm²).

The concentration of the type 1 collagen from Sigma in the stock solution (0.1 N acetic acid, neutralized with NaOH pH 7.0; filtered) is 400 μg/ml. This solution was diluted 1:10 in PBS for coating (10 μg/cm²).

Other concentrations of the collagen and other collagen types also proved to be suitable.

The concentration of the recombinant silicatein (silicatein a; S. domuncula) in the stock solution (PBS; filtered) is 40 μg/ml. This solution is diluted 1:10 in PBS for coating (10 μg/cm²).

Other concentrations of the silicatein also proved to be suitable. Silicatein β can also be used as enzyme for the modification just as silicatein α.

The stock solution of tetraethylorthosilicate (tetraethoxysilane, TEOS; Aldrich) had a concentration of 5 mM. TEOS is dissolved in dimethylsulfoxide in a stock solution of usually 500 mM and subsequently diluted down to the desired end concentration.

Other concentrations of TEOS and other substrates (silicic acids, monoalkoxysilanetriols, dialkoxysilanediols, trialkoxysilanols or tetraalkoxysilanes for the production of silica and monoalkoxysilanediols, monoalkoxysilanols, dialkoxysilanols, alkylsilanetriols, arylsilanetriols or metallosilanetriols, alkylsilanediols, arylsilanediols or metallosilanediols, alkylsilanols, arylsilanols or metallosilanols, alkylmonoalkoxysilanediols, arylmonoalkoxysilanediols or metallomonoalkoxysilanediols, alkylmonoalkoxysilanols, arylmonoalkoxysilanols or metallomonoalkoxysilanols, alkyldialkoxysilanols, aryldialkoxysilanols or metallodialkoxysilanols, alkyltrialkoxysilanes, aryltrialkoxysilanes or metallotrialkoxysilanes for the production of silicones) have also proven to be suitable.

### 2.9. Determination of the Concentration of DNA

The total DNA in the batches can be determined with the aid of methods that are state of the art, e.g., the PicoGreen assay. To this end, PicoGreen dsDNA quantitation reagent (molecular probes) is diluted 1:200 in TE buffer (10 mM tris/HCl pH 7.4, 1 mM EDTA). The PicoGreen solution is subsequently mixed 1:1 (100 μl: 100 μl) with the samples (cells suspended in TE buffer). The batches are allowed to stand in the dark for 5 minutes and then measured with the aid of a fluorescence ELISA plate reader (e.g., Fluoroskan version 4.0) at an excitation of 485 nm and emission of 535 nm. A calibration curve with calf's thymus DNA was recorded as comparison standard.

### 2.10. Demonstration of the Mineralization with Alizarin Red S

The formation of calcium phosphate by osteoblasts such as, e.g., SaOS-2 cells can be measured according to the method of Stanford et al. (J. Biol. Chem. 270:9420-9428, 1995) or other methods that are state of the art. The cells are fixed 1 hour at 4° C. in 100% ethanol, then briefly washed with distilled H₂O and stained with 40 mM alizarin red S solutions (pH 4.2; Sigma company) for 10 minutes at room temperature under gentle agitation. The cells are then washed several times with distilled H₂O and with 1×PBS (DULBECCO). The cells are then incubated in 100 μl/cm² of 10 wt. % cetylpyridinium chloride (CPC), 10 mM sodium phosphate (pH 7.0) for 15 minutes at room temperature under gentle agitation. An aliquot from the supernatants is diluted 10 times in 10% CPC, 10 mM sodium phosphate (pH 7.0) and the absorption measured at 562 nm. The moles of bound alizarin red-S can be determined with a calibration curve. The obtained values are related to the total DNA amounts determined in parallel cultures.

### 3. USES

A further aspect of the invention are the uses of the method cited below for the production of bioactive surfaces by enzymatic modification of molecules or molecular aggregates on surfaces by means of amorphous silicon dioxide (silica) with silicatein α, silicatein β or related polypeptides as well as of the products obtained.

1. The use of the method for the production of bioactive surfaces by enzymatic modification of molecules or molecular aggregates on surfaces amorphous silicon dioxide (silica) as well as of the obtained products in the cell culture in tissue engineering or in medicinal implants.

2. The use of the method as well as of the products obtained for increasing the growth (of cells and cell cultures in general and especially of fibroblasts and bone-building cells/osteoblasts) as well as the increasing of the mineralization (of bone-building cells/osteoblasts).

3. The use of the method as well as of the obtained products to produce a matrix that favors or furthers the depositing of calcium phosphate or apatite.

4. The use of the method as well as of the obtained products to produce a stable connection in particular between bones and implants wherein the following occur: a) a migration of Ca²⁺ and PO₄³⁻ groups from the solution, the medium or a body fluid or released from cells or formed under the participation of cellular enzymes (such as, e.g., the release of phosphate from β-glycerophosphate with the aid of the alkaline phosphatase associated with the osteoblast membrane) onto the SiO₂ layer on the surface with the deposition of calcium phosphate, (b) the growth of the amorphous calcium phosphate layer produced by the inclusion of more soluble calcium and phosphate, and (c) crystallization of the amorphous calcium phosphate layer by the inclusion of hydroxide anions, carbonate anions and fluoride anions (contained, e.g., in and from body fluids) under formation of a mixed apatite material consisting of hydroxylapatite, carbonateapatite, and fluoroapatite.

5. The use of the method as well as of the obtained products for improving the biocompatibility of medical implants.

6. The use of the method for producing coatings for biomaterials, plastics, metals, metal oxides and other materials for furthering the cellular adhesion to these materials as a prerequisite for the tissue integration with the surface of implants.

7. The use of the method to produce SiO₂ layers on surface-bound molecules or molecular aggregates of implants in order to reduce immunological reactions of the receiving organism such as antigen-antibody reactions or the bonding of components of the complement system to the implant surface.

1. A method for producing bioactive surfaces by enzymatic modification of molecules or molecular aggregates on surfaces with amorphous silicon dioxide (silica) and/or silicones comprising an enzymatic modification by a polypeptide, wherein the polypeptide comprises an animal, bacterial, vegetable or fungal silicatein α or silicatein β domain exhibiting at least 25% sequence identity with the sequence shown in SEQ ID No. 1 or SEQ ID No. 3, or an animal, bacterial, vegetable or fungal silicase domain exhibiting at least 25% sequence identity with the sequence shown in SEQ ID No. 5.

2. The method according to claim 1, characterized in that silicic acids, monoalkoxysilanetriols, dialkoxysilanediols, trialkoxysilanols or tetraalkoxysilanes are used as substrate for the enzymatic modification.

3. The method according to claim 1, characterized in that monoalkoxysilane diols, monoalkoxysilanols, dialkoxysilanols, alkylsilanetriols, arylsilanetriols or metallosilanetriols, alkylsilanediols, arylsilanediols or metallosilanediols, alkylsilanols, arylsilanols or metallosilanols, alkylmonoalkoxysilanediols, arylmonoalkoxysilanediols or metallomonoalkoxysilanediols, alkylmonoalkoxysilanols, arylmonoalkoxysilanols or metallomonoalkoxysilanols, alkyldialkoxysilanols, aryldialkoxysilanols or metallodialkoxysilanols, alkyltrialkoxysilanes, aryltrialkoxysilanes or metallotrialkoxysilanes are used as substrate for the enzymatic modification.

4. The method for producing bioactive surfaces by enzymatic modification of molecules or molecular aggregates on surfaces according to claim 1, wherein the surface is the surface of glass, metals, metal oxides, plastics, or biopolymers.

5. The method for producing bioactive surfaces by enzymatic modification of molecules or molecular aggregates on surfaces according to claim 1, wherein the molecules or molecular aggregates are biopolymers.

6. The method for producing bioactive surfaces by enzymatic modification of molecules or molecular aggregates on surfaces according to claim 1, wherein the molecules or molecular aggregates are collagen.

7. The method for producing bioactive surfaces by enzymatic modification of molecules or molecular aggregates on surfaces according to claim 6, wherein the molecules or molecular aggregates are a collagen from a marine sponge.

8. The method for producing bioactive surfaces by enzymatic modification of molecules or molecular aggregates on surfaces according to claim 1, wherein the polypeptide of the silicatein α or silicatein β from Suberites domuncula in accordance with SEQ ID No. 1 or SEQ ID No. 3 or a polypeptide homologous to it that exhibits at least 25% sequence identity with the sequence shown in SEQ ID No. 1 or SEQ ID No. 3 in the amino acid sequence of the silicatein α or silicatein β domain is made available in vivo, in a cell extract or cell lysate or in purified form.

9. The method for producing bioactive surfaces by enzymatic modification of molecules or molecular aggregates on surfaces according to claim 1, wherein the polypeptide of the silicase from Suberites domuncula in accordance with SEQ ID No. 5 or a polypeptide homologous to it that exhibits at least 25% sequence identity with the sequence shown in SEQ ID No. 5 in the amino acid sequence of the silicase domain is made available in vivo, in a cell extract or cell lysate or in purified form.

10. A silicic acid-containing structure or surface obtained by producing bioactive surfaces by enzymatic modification of molecules or molecular aggregates on surfaces with amorphous silicon dioxide (silica) and/or silicones comprising an enzymatic modification by a polypeptide, wherein the polypeptide comprises an animal, bacterial, vegetable or fungal silicatein α or silicatein β domain exhibiting at least 25% sequence identity with the sequence shown in SEQ ID No. 1 or SEQ ID No. 3, or an animal, bacterial, vegetable or fungal silicase domain exhibiting at least 25% sequence identity with the sequence shown in SEQ ID No. 5.

11. A method for promoting the growth, activity and/or the mineralization of cells and/or cell cultures comprising a) producing bioactive surfaces by enzymatic modification of molecules or molecular aggregates on surfaces with amorphous silicon dioxide (silica) and/or silicones comprising an enzymatic modification by a polypeptide, wherein the polypeptide comprises an animal, bacterial, vegetable or fungal silicatein α or silicatein β domain exhibiting at least 25% sequence identity with the sequence shown in SEQ ID No. 1 or SEQ ID No. 3, or an animal, bacterial, vegetable or fungal silicase domain exhibiting at least 25% sequence identity with the sequence shown in SEQ ID No. 5 and b) bringing the cells and/or cell cultures in contact with the bioactive surface obtained in step a).

12. The method for promoting the growth, activity and/or the mineralization of cells and/or cell cultures according to claim 11, wherein the cells are selected from osteoblasts or cells similar to osteoblasts.

13. A method for tissue engineering or producing medical implants wherein said method comprises the step of producing bioactive surfaces by enzymatic modification of molecules or molecular aggregates on surfaces with amorphous silicon dioxide (silica) and/or silicones comprising an enzymatic modification by a polypeptide, wherein the polypeptide comprises an animal, bacterial, vegetable or fungal silicatein α or silicatein β domain exhibiting at least 25% sequence identity with the sequence shown in SEQ ID No. 1 or SEQ ID No. 3, or an animal, bacterial, vegetable or fungal silicase domain exhibiting at least 25% sequence identity with the sequence shown in SEQ ID No. 5.


SEQUENCE LISTING (12 sequences)

SEQ ID NO: 1 - protein, 330 aa, Suberites domuncula
MLVTVVVLGLLGFASAAQPKFEFVEEWQLWKSTHSKMYESQLMELERHLTWLSNKKYIEQHNVNSHIFGFTLAMNQFGDLSELEYANYLGQYRIEDKKSGNYSKTFQRDPLQDYPEAVDWRTKGAVTAVKDQGDCGASYAFSAMGALEGANALAKGNAVSLSEQNIIDCSIPYGNHGCHGGNMYDAFLYVIANEGVDQDSAYPFVGKQSSCNYNSKYKGTSMSGMVSIKSGSESDLQAAVSNVGPVSVAIDGANSAFRFYYSGVYDSSRCSSSSLNHAMVVTGYGSYNGKKYWLAKNSWGTNWGNSGYVMMARNKYNQCGIATDASYPTL

SEQ ID NO: 2 - DNA, 1214 bp, Suberites domuncula
aggatagaaagtctacaatctgtaaggacaatgcttgtcacagtggtagtactgggtctactggggtttgcttctgcagcccagcccaagtttgaatttgtagaagaatggcagctgtggaagtccactcactctaagatgtacgagtcacagttaatggaactcgaaagacatctgacgtggctctccaataagaaatatatcgagcaacacaatgtcaactcacacattttcggttttactctggcaatgaaccagtttggagatctgagtgaattggagtatgctaactatcttggccagtatcgcattgaggataaaaaatctggcaactactcaaagacttttcagcgtgatcctctacaggactaccctgaagctgtagactggagaaccaaaggagctgtcacggctgtcaaggaccagggagactgtggtgctagctatgctttcagtgctatgggtgctttggagggtgctaatgctttagccaagggaaatgcagtatctctcagtgaacagaacatcattgattgctcgattccttacggtaaccacggttgtcatggaggcaatatgtatgatgcttttttgtatgtcatcgctaacgagggggtcgatcaggacagtgcatatccatttgtaggaaagcaatccagctgcaactataatagtaaatacaaaggtacatcaatgtcggggatggtgtcaatcaaaagtggtagtgagtctgacttacaagcagctgtttcaaacgttggccctgtatctgttgctattgatggtgctaacagtgccttcaggttttactacagtggtgtctatgactcatcacgatgctctagtagtagtcttaaccacgcaatggtagtcactggatacggatcatacaatgggaaaaaatactggctggccaagaatagctggggaactaactggggtaacagtggctatgtgatgatggctcgcaacaagtacaaccagtgtggaattgctaccgatgcatcttatcccaccctataaacttatatatatatagtcttagaaacattatccttttctttacccttgtctctataggccatagagtgattgtaggctgtttgcatttgatgactgtatataccctatcattttttgtgattctatctgattaaaaatcccatacccgaccaaaccatcaatttatcaaatcatgaaaaaaaaaaaaaaaa

SEQ ID NO: 3 - protein, 383 aa, Suberites domuncula
MSALKFVVALCVVHTSLGIAESVGKSKTAGLSDDGNYTAVTKSVRLTPVLEFEEDWKQWTTDHHKVYSDVRERVDKYTVWRANKEYIDQHNQNAQRLGYTLKMNKFGDLTTKEFIEGYHCVQDYQPTNASHLNKKHKTHAFVDYGDFVRGGTGEGVRGVGNMPETMDWRTSGVVTKVKDQLRCGSSYAFSAMASLEGINALSYGSLVTLSEQNIVDCSVTYGNHGCACGDVNRALLYVIENDGVDTWKGYPSGGDPYRSKQYSCKYERQYRGASARGIVSLASGDENTLLTAVANSGPVSVYVDATSTSFQFYSDGVLNVPYCSSSTLSHALVVIGYGKYSGQDYWLVKNSWGPNWGVRGYGKLARNKGNKCGIATAASFPTL

SEQ ID NO: 4 - DNA, 1372 bp, Suberites domuncula
acttagtatattatggtagtgtacaatacctatctccatagaggcatgcataactaggtttattgaatagctgctggcacaatttgttctcaagttggtgctattagatttgtgttctagaatgtcagcattgaagtttgtagttgccttgtgtgtagttcacacaagcttaggaatagctgagtcagttggtaagagcaagactgcaggcctaagtgacgatggcaactacacagctgtcaccaaatctgtaagactgactccagttctagagtttgaggaagattggaagcaatggacaactgatcatcacaaggtctactctgatgtgagggagagagtggacaagtacactgtatggagagctaataaagagtacattgatcaacacaaccagaacgcacagagattgggatacacactcaaaatgaacaaatttggagatttgactaccaaggagttcattgaaggctatcactgtgttcaggactaccaacctaccaatgcatcacatttgaataagaaacacaaaacgcacgcgtttgtcgactatggtgactttgtgaggggtggaactggtgagggtgtgaggggtgtaggaaacatgccggagactatggactggagaacttctggagttgtcacaaaagttaaagatcagcttcgttgtggtagcagctatgcgttctctgccatggcttcattggaaggaataaatgctctttcctacggatctttggtgacactcagtgaacaaaacattgtagactgctcggttacctatggcaaccatggttgcgcctgtggtgatgtaaaccgtgctctactgtatgtgatagagaatgatggcgttgacacgtggaagggttatccttctggtggggatccttatcgatcaaagcaatactcttgcaaatacgagagacagtatcgtggggcctctgctagaggtatagtcagtctagctagtggtgatgagaacacattgttgacagcagtagctaactctggaccagtgagtgtgtatgtggacgctacttcaacatccttccagttttacagtgatggagtgttgaatgttccctattgctcctctagcacgctgagtcatgccttggttgtcattggttacgggaagtacagcggacaagattactggcttgttaaaaacagctggggtcctaactggggagtgcggggctatgggaagttggcaagaaacaagggcaacaaatgtggaatagccacagcggctagtttcccaacattatgacactttagttgatcaaacaattaatcataaattattacaacatgtagtataatgatgcccccccattgctcaatagcttatctttgaacaagaaaaaaa

SEQ ID NO: 5 - protein, 379 aa, Suberites domuncula
MSAILKRNVPIQRVGLPLTSYVSRWASALPTRTHPFYKLVDDSTTPVTRSTLLSAHMVDTLLDENQQSRHENQHTDTSYKMYQGLKFVVKTLFTPSKCHRHFSTSAHLSAMGRHQSPINIITSSTTKGPSLKPLKFSKSWDKPVIGTVKDTGYYLKFAPESAAEKCTLHTYNGEYILDHFHYHWGKKDGEGAEHFIDGKQYDIEFHFVHKKVGLTDPDARDAFAVLGVFGKADPRLKINGIWELLSPSTVLTVDSTRNVADVVPSKLLPSARDYFHYEGSLTTPTYGEVVHWFVLNEPIAVPSEYLSALRQMQADKEGTVIDSNYRELQEVHNRPVQRFKSDEQGRGEFDDISKNEDIVEDLSKLSGNFIRELVRKIYW

SEQ ID NO: 6 - DNA, 1405 bp, Suberites domuncula
gaattcggcacgagggacaactttgcataacttttactgtccatgtttaacgtttagatctagtactagtagtctacaagaacaactgtcaacaactgtcagattatgtgtataaaccaagatgtctgcaattcttaagagaaacgtacctatccaaagagtcggtctcccactgacctcctatgtcagtagatgggcttctgctctgcccaccaggacccatcctttttacaagttggttgatgacagtaccaccccagtgacaaggtctactcttctcagtgctcatatggttgacaccttgctagatgagaaccagcagagcagacatgaaaaccaacacacagacacgtcttacaaaatgtaccagggattaaaatttgttgtaaagacgctgtttactccatcgaaatgccaccgtcacttctccacatcagctcatttgtctgccatgggtcgacatcaatcccccatcaatataatcacctccagtacgaccaaaggaccgtcattgaaaccgttaaaatttagcaagagttgggacaagccagtaatcggcaccgtcaaagatactggctattatcttaaatttgcaccagaatctgcagcagagaagtgcacattgcatacgtacaatggtgaatatatcctagatcatttccattatcactgggggaagaaggatggggaaggagcagagcatttcatcgatggaaaacaatacgacatcgagttccactttgtacataaaaaggttgggttgactgatccagatgctagagacgcttttgctgttttgggcgtttttggaaaggccgaccctcgtttgaagatcaatggaatctgggagctactctcaccgtcaactgtcctgactgtcgactcaacacgaaacgtcgctgatgttgttccctctaagcttctcccaagtgccagagactattttcactatgaaggttctttgaccacacctacgtatggtgaggttgtgcactggtttgttctcaatgaacccatagctgtccctagtgagtatctgtcagctctgagacagatgcaagctgacaaagaaggtactgtgattgactcaaactatcgagagcttcaagaagtccacaatcgacctgtgcaacgatttaagagtgatgagcaagggagaggagaatttgacgatatttctaagaatgaggacattgtggaggacttgtctaaattgtctggtaactttattagagagctggtcaggaagatatattggtgacctttttctacacttgttagagttttaggccagaatacatttcatcatttggactgttattttgtgtacactgcttagcagtttatataaacactacaatgccattattataatatagccaatgctgtgatttgaaaaaaaaaa

SEQ ID NO: 7 - protein, 268 aa, Suberites domuncula
MVAISGQAIGPQGPRGLPGRDGRDGQPGSPGSDGQSGSPGRDGRDGLTGPQGVIGEPGPANGPQGPRGLQGTRGEPGSQGPAGQPGPTSGGVVYTRWGKSTCPSVAGTQLVYAGRAGGSEHRDAGAANHLCMPLDPEYTLQHRSGVQGSMYVYGTEYQSPIRGTDNHNVPCAVCSTSTRAQLLMIPAKTSCPTSWTREYYGYLMSQHRSHHPSMYECVDKIKNLSQGVLLTPMVLFSIHVEATCNGMQCPPYNNYKELNCVVCTKRSF

SEQ ID NO: 8 - DNA, 1038 bp, Suberites domuncula
ggcacgaggttaaacagctctcgtgaaaaacatggaaccaaagacctactactctttgctgcagttacactactagtaatgattgtggcaatatcaggtcaggctataggacctcaaggcccacgtggactaccaggaagagatggtcgtgatggacaacctgggtcacctggtagtgatggacaatctggatcacctggtagagatggaagagatggattgactggccctcaaggtgtaataggagaaccaggaccagccaatggaccacaaggacccagaggactacaggggacaaggggagaaccagggtcacaaggacctgctggtcaaccaggacccacgagtggaggtgttgtctacacaaggtgggggaagagcacttgtcctagtgttgctgggactcaactggtatatgctggaagagctggcgggagtgaacacagagatgctggggcagccaaccatctctgtatgcctttagaccctgagtacaccctacaacatcgtagtggagtgcaaggcagtatgtatgtatatggaacagaataccaatctcctattagaggaacagataatcacaacgttccatgtgctgtgtgttcaacatctactcgagcccagctcttaatgattccagctaagacatcctgtccgacatcatggactagagagtactatggctatctcatgtctcaacatcgttcccatcatccttcaatgtatgaatgcgttgacaagatcaagaatctgtcccagggagttctgctaacaccaatggtgctcttttctatccacgttgaagctacctgcaatggtatgcaatgtcctccatacaataactacaaagaactgaactgtgttgtctgcacaaagtaaacttattgacatagtttgtattgcgtgcatgcagtcattaatgtgataggtactgccagtgtgaaaatagagattattgtgggtgtgtgcgtgtgcgtgttttgtgcatgtgtgctgtttttcaatatatcttttattaatataaaaaaaaaaaaaaaa

SEQ ID NO: 9 - RNA, 30 nt, Suberites domuncula
accaggacacccgaagcgagacggagaacc

SEQ ID NO: 10 - DNA, 40 bp, Suberites domuncula
tattctagaattatagggtgggataagatgcatcggtagc

SEQ ID NO: 11 - DNA, 39 bp, Suberites domuncula
tatccatggtggcaatatcaggtcaggctataggacctc

SEQ ID NO: 12 - DNA, 38 bp, Suberites domuncula
tataagcttcgctttgtgcagacaacacagttcagttc